(19) United States
(12) Patent Application Publication
(10) Pub. No.: US 2020/0392296 A1
(43) Pub. Date: Dec. 17, 2020

(54) Nano coordination polymer and preparation method and application thereof

(71) Applicant: Central South University, Changsha (CN)

(72) Inventors: Wenhu Zhou, Chandsha (CN); Ying Peng, Chandsha (CN); Mengzhen Yu, Chandsha (CN); Jinsong Ding, Hunan (CN)

(73) Assignee: Central South University

(21) Appl. No.: 17/006,785

(22) Filed: Aug. 28, 2020

(30) Foreign Application Priority Data
Aug. 29, 2019 (CN) 201910805848.4

(51) Int. Cl. C08G 83/00; A61K 9/51; A61K 47/02; A61K 47/20; A61P 35/00; A61K 33/34

(52) U.S. Cl. CPC C08G 83/008; A61K 9/513; A61K 33/34; A61K 47/20; A61P 35/00; A61K 47/02

(57) ABSTRACT

According to a nano coordination polymer and a preparation method and application thereof, the nano coordination polymer includes: ditiocarb sodium and copper, wherein the ditiocarb sodium and the copper form a polymer through coordination. The nano coordination can be wrapped by a targeting ligand through electrostatic interaction to form a core-shell structure. The preparation method includes steps of: mixing ditiocarb sodium with a stabilizer to obtain a mixed solution, and dripping CuCl₂ into the mixed solution through a constant flow pump to obtain a polymer; and dripping a targeting ligand into the polymer through the constant flow pump; then stirring to obtain the nano coordination polymer. The nano coordination polymer will interfere with the p97 pathway, cause the accumulation of ubiquitinated proteins, and then lead to impaired protein degradation, and finally induce cell apoptosis.

### CROSS REFERENCE OF RELATED APPLICATION

The present invention claims priority under 35 U.S.C. 119(a-d) to CN 201910805848.4, filed Aug. 29, 2019.

### BACKGROUND OF THE PRESENT INVENTION

### Field of Invention

The present invention relates to a field of nano-biotechnology, and more particularly to a nano coordination polymer and a preparation method and application thereof.

### Description of Related Arts

Malignant tumor is the major disease that seriously threaten the human lives with high morbidity and mortality. Currently, there is no effective treatment. Most tumors are characterized by malignant proliferation. Compared with normal tissues, the structure of neovascularization in tumor tissues is incomplete, and there are larger gaps between vascular endothelial cells. Therefore, the permeability of tumor blood vessels is higher than that of normal tissues, which facilitates the retention of nanomedicine in the tumor site. In addition, due to the activation and expression of tumor-related genes, some specific receptors are highly expressed on tumor cell membranes. For example, MDA-MB-231 cells (M231 cells, human triple-negative breast cancer cells) highly express CD44 receptors, and the expression of folate receptors (FR) is up-regulated in Hela cells (human cervical cancer cells). The above is helpful for targeted drug delivery. Currently, chemotherapy is one of the main methods for treating tumors. Commonly used chemotherapy drugs include doxorubicin, paclitaxel, cisplatin and so on. However, these drugs are expensive and can cause serious side effects. Therefore, constructing a drug delivery system with high efficiency, low toxicity and low price has certain clinical practical significance.

### SUMMARY OF THE PRESENT INVENTION

In order to solve the above problems, an object of the present invention is to provide a nano coordination polymer, which can interfere with the P97 pathway, cause the accumulation of ubiquitinated proteins, and then lead to impaired protein degradation, and finally induce cell apoptosis. The nano coordination polymer can be easily prepared, and can be used to prepare tumor-targeted drugs.

Accordingly, in order to accomplish the above objects, the present invention provides a nano coordination polymer, which includes ditiocarb sodium and copper, wherein the ditiocarb sodium and the copper form a polymer through coordination.

Preferably, a molar ratio of the ditiocarb sodium and the copper is 2:1.

Preferably, the nano coordination polymer can be wrapped by a targeting ligand through electrostatic interaction to form a core-shell structure.

Preferably, the targeting ligand is a hyaluronic acid, synthetic polypeptide, a folate-modified hydrophilic polymer, or a tumor-targeted nucleic acid aptamer.

Accordingly, the present invention also provides a preparation method of the above nano coordination polymer, comprising steps of:

S1: mixing ditiocarb sodium with a stabilizer to obtain a mixed solution, and dropping CuCl₂ into the mixed solution to obtain the nano coordination polymer; and

S2: dripping a targeting ligand, which is in a solution form, into the nano coordination polymer; then stirring to obtain the tumor targeting nano coordination polymer.

Preferably, the stabilizer is polyethylene glycol (PEG), polyvinyl alcohol (PVA), polyvinylpyrrolidone (PVP) or Tween.

Preferably, a concentration of the stabilizer is 0.4 wt %-1 wt %.

Accordingly, the present invention also provides a method for preparing a tumor targeted therapy drug, comprising adding the above nano coordination polymer into the tumor drug.

Accordingly, the present invention also provides a method for preparing a targeted pro-apoptotic drug for malignant tumor, comprising adding the above nano coordination polymer into the targeted pro-apoptotic drug.

Accordingly, the present invention also provides a method for preparing a targeted pro-apoptotic drug for triple-negative breast cancer cells, comprising adding the above nano coordination polymer into the targeted pro-apoptotic drug.

Preferably, the drug is an external preparation, an oral preparation, or an injection.

Preferably, the external preparation is a gel for external use.

The oral preparation is granules, tablets, oral solutions, or the like containing the nano coordination polymer.

The injection is an intravenous injection containing the nano coordination polymer.

Compared with the prior art, the present invention has the following beneficial effects:

1. The present invention provides the nano coordination polymer, wherein the ditiocarb sodium and copper ions form a complex (CuET). CuET will interfere with the p97 pathway, cause the accumulation of ubiquitinated proteins, and then lead to impaired protein degradation, and finally induce cell apoptosis. A prerequisite for the ditiocarb sodium to exert an anti-tumor effect is to form the complex with the copper ions. However, materials such as liposomes and micellar materials are also used to co-carry copper ions and disulfiram, but there are problems that the related preparation technology is relatively complicated, and the loading of disulfiram and copper ions is low. The nano coordination polymer of the present application is an amorphous nano-hybrid material composed of metal ions or metal clusters and an organic ligand cross-linked by metal coordination bonds. The ditiocarb sodium can form nanoparticles through coordination and cross-linking of sulfhydryl groups in its structure with thiophilic copper ions. The nano coordination polymer can be formed directly through coordination without additional inorganic or organic carrier. The preparation method of the co-loaded ditiocarb sodium and copper ions is simple and efficient.

2. The present invention provides the nano coordination polymer formed by the ditiocarb sodium and the copper ions. The coordination polymer has a nanometer-scale particle size and can be passively targeted to the tumor site through enhanced permeability and retention effect (EPR effect), thereby increasing a cumulative amount of the drug in the tumor site, and reducing toxic side effects. The nano coordination polymer can be further modified by hyaluronic acid. The hyaluronic acid is the specific ligand of CD44 receptor, which can bind to the highly expressed CD44 receptor on tumor cells (such as human breast cancer cell M231, human liver cancer cell HepG2, human gastric cancer cell SGC-7901, and human bladder cancer cell T24) to increase the uptake efficiency of coordination polymers by specific tumor cells, thereby further enhancing therapy efficacy.

3. The present invention provides the preparation method of the nano coordination polymer, which is simple and controllable.

4. The present invention provides applications of the nano coordination polymer in preparation of tumor-targeted drugs. The nano coordination polymer can target and enrich in tumor lesions to inhibit tumor growth by promoting tumor cell apoptosis, which does no harm to liver, spleen, lung, kidney, etc., and can provide basis and ideas for tumor treatment. Nano polymers can be passively enriched in all solid tumor tissues (through the recognized EPR effect), and can actively target all CD44 over-expressed tumor cells. CuET will interfere with the p97 pathway, cause the accumulation of ubiquitinated proteins, and then lead to impaired protein degradation, and finally induce cell apoptosis.

### BRIEF DESCRIPTION OF THE DRAWINGS

In order to explain the embodiments of the present invention or the technical solutions in the prior art more clearly, the drawings described in the embodiments will be briefly introduced below. Obviously, the drawings in the following description are only some of the embodiments of the present invention. For those of ordinary skill in the art, other drawings may be obtained in view of these drawings without creative work.

FIG. 1 illustrates particle sizes and Zeta-potential changes of CuET NPs and CuET@HA NPs according to embodiments 1 and 2 of the present invention;

FIG. 2 is a transmission electron microscope image of CuET@HA NPs according to the embodiment 2 of the present invention.

FIG. 3 is a UV absorption spectrum of CuET NPs and CuET@HA NPs according to the embodiments 1 and 2 of the present invention:

FIG. 4 is an infrared absorption spectrum of DTC, CuET NPs and CuET@HA NPs according to the embodiments 1 and 2 of the present invention:

FIG. 5 is an X-ray photoelectron spectrogram of CuET NPs and CuET@HA NPs according to the embodiments 1 and 2 of the present invention;

FIG. 6 illustrates a molar ratio of DTC to Cu²⁺ in CuET NPs and CuET@HA NPs according to the embodiments 1 and 2 of the present invention;

FIG. 7 illustrates particle size changes curve of CuET NPs and CuET@HA NPs in PBS and DMEM complete media according to the embodiments 1 and 2 of the present invention:

FIG. 8 is a release curve diagram of CuET NPs under different conditions according to the embodiment 1 of the present invention;

FIG. 9 is a release curve diagram of CuET@HA NPs under different conditions according to the embodiment 2 of the present invention:

FIG. 10 is images of CuET NPs and CuET@HA NPs under a confocal laser microscope according to an embodiment 3 of the present invention:

FIG. 11 illustrates cell viability of CuET NPs and CuET@HA NPs according to an embodiment 4 of the present invention;

FIG. 12 is IC₅₀ results of CuET NPs and CuET@HA NPs according to the embodiment 4 of the present invention;

FIG. 13 illustrates results of ubiquitinated protein expression after incubating CuET NPs having concentrations of 0.1, 0.2, 0.5 and 1 μM for 24 h with M231 cells according to an embodiment 5 of the present invention;

FIG. 14 illustrates in-vivo distribution of a drug according to an embodiment 6 of the present invention, wherein part A is fluorescence distribution in mice at 1 h and 24 h after tail intravenous injection; part B is fluorescence distribution of heart, liver, spleen, lung, kidney and tumor after the mice were sacrificed 24 h after the injection;

FIG. 15 illustrates in-vivo efficacy evaluation of a nano coordination polymer according to an embodiment 7 of the present invention, wherein part A is a graph of tumor volume change in each group; part B is a graph of weight changes of mice in each group; part C is an image of solid tumors in each group on the 14th day;

FIG. 16 is images of H&E staining, Tunel staining and Caspase-3 immunofluorescence staining of tumor tissues in each group according to the embodiment 7 of the present invention;

FIG. 17 illustrates pathological sections of heart, liver, spleen, lung and kidney of each group of mice according to an embodiment 8 of the present invention.

### DETAILED DESCRIPTION OF THE PREFERRED EMBODIMENT

The following embodiments are used to illustrate the present invention, rather than limiting the scope of the present invention. Without departing from the spirit and essence of the present invention, modifications or substitutions made to the methods, steps or conditions of the present invention fall within the scope of the present invention.

If not specified, the technical means used in the embodiments are conventional methods well-known to those skilled in the art. If not specified, the reagents used in the embodiments are all commercially available.

If not specified, the percentage sign “%” involved in the present invention refers to mass percentage: but the percentage of the solution, unless otherwise specified, refers to the number of grams of solute contained in 100 ml of the solution.

The weight part of the present invention can be a weight unit known in the art such as μg, mg, g, kg, etc., or a multiple thereof, such as 1/10, 1/100, 10 times, 100 times, and the like.

In the following embodiments, detailed information of used instruments and manufacturers thereof are shown in Table 1:

| Instrument | Manufacturer |
|---|---|
| CP225D electronic balance | Sartorius, Germany |
| BP224S electronic balance | Sartorius, Germany |
| XW-80A vortex mixer | Shanghai Qingpu Huxi Analytical |
| | Instrument Factory |
| handheld centrifuge | Scilogex, USA |
| DF-101S constant temperature heating | Gongyi Yuhua Instrument |
| magnetic stirrer | Co., Ltd. |
| UV-2600 UV Spectrophotometer | Shimadzu Corporation, Japan |
| TGL16M low temperature high speed | Changsha Yingtai Instrument |
| centrifuge | Co., Ltd. |
| TD4A benchtop low speed centrifuge | Changsha Yingtai Instrument |
| | Co., Ltd. |
| SHA-B water bath constant | Changzhou Aohua Instrument |
| temperature oscillator | Co., Ltd. |
| MIN4-UVF pure water machine | Hunan Colton Water Co., Ltd. |
| UV-2600 UV spectrophotometer | Shimadzu Corporation, Japan |
| LC-2010A high performance liquid | Shimadzu Corporation, Japan |
| chromatograph | |
| Infinite M200PRO multifunctional | Austrian TECAN Company |
| microplate reader | |
| Nano-ZS90 particle size analyzer | British Malvern Instruments |
| | Co., Ltd. |
| Tecnai G2 F20 transmission electron | American FEI Company |
| microscope | |
| TGL20M desktop high-speed | Changsha Yingtai Instrument |
| refrigerated centrifuge | Co., Ltd. |
| 1 × 70 inverted fluorescence | Olympus Japan |
| microscope | |
| Forma Series II CO₂ cell incubator | American Thermo Fisher |
| | Company |
| SW-CJ-2FD vertical purification | Suzhou Purification Equipment |
| workbench | Co., Ltd. |
| DSX-30L autoclave | Shanghai Shen'an Medical |
| | Equipment Factory |
| 4° C. refrigerator | China Haier Medical Refrigerator |
| −20° C. refrigerator | China Haier Medical |
| | Cryopreservation Refrigerator |
| −80° C. refrigerator | China Haier Medical |
| | Cryopreservation Refrigerator |
| FACSVerse flow cytometer | American BD Company |
| Q5000 trace ultraviolet | |
| spectrophotometer | American QUAWELL company |
| PCR instrument | American Thermo Fisher |
| | Company |
| CFX-Connect fluorescence | American BIO-RAD Company |
| quantitative PCR instrument | |
| 7700X ICP-MS | Agilent Corporation of Japan |
| IVIS III small animal in-vivo imager | PerkinElmer, USA |
| JJ-12J dehydration machine | Wuhan Junjie Electronics |
| | Co., Ltd. |
| JB-P5 embedding machine | Wuhan Junjie Electronics |
| | Co., Ltd. |
| RM2016 pathology slicer | Shanghai Leica Instruments |
| | Co., Ltd. |
| KD-P tissue spreader | Zhejiang Jinhua Kedi Instrument |
| | Equipment Co., Ltd. |

In the following embodiments, names and manufacturers of main reagent used are shown in Table 2:

| Reagent | Manufacturer |
|---|---|
| ditiocarb sodium | American Sigma Company |
| copper chloride dihydrate | Aladdin Reagent Co., Ltd. |
| polyvinylpyrrolidone (30 KD) | Sinopharm Chemical Reagent Co., |
| | Ltd. |
| hyaluronic acid (120 KD-250 | Hunan Huateng Pharmaceutical Co., |
| KD) | Ltd. |
| Rhodamine B | Sinopharm Chemical Reagent Co., |
| | Ltd. |
| chlorin | Aladdin Reagent Co., Ltd. |
| sodium chloride | Sinopharm Chemical Reagent Co., |
| | Ltd. |
| penicillin mixture (100 × double | Beijing Soleibao Technology Co., |
| antibody) | Ltd. |
| pancreatin | Beijing Soleibao Technology Co., |
| | Ltd. |
| fetal bovine serum | American GIBCO Company |
| DMEM medium | American GIBCO Company |
| 4′,6-diamidino-2- | American Sigma Company |
| phenylindole (DAPI) | |
| methyl thiazolyl tetrazolium | American Sigma Company |
| (MTT) | |
| Ubiquitin antibody | Abkang Trading Co., Ltd. |
| Caspase-3 antibody | Hunan Aijia Biotechnology Co., Ltd. |

### Embodiment 1

A nano coordination polymer comprises a polymer formed by ditiocarb sodium and copper through coordination.

A preparation method of the nano coordination polymer of the embodiment 1 comprises steps of:

(1) adding 6 mL 2 mM ditiocarb sodium (DTC) into a 20 mL beaker, adding 184 μL 20 wt % polyvinylpyrrolidone (PVP), and stirring at room temperature for 2 min to obtain a mixed solution; and

(2) using a 5 mL syringe to take 3 mL 2 mM CuCl₂ and dripping into the mixed solution of the step (1) with a constant flow pump at a dripping rate of 20 μL/min, thereby obtaining a ditiocarb sodium/copper nano coordination polymer (CuET NPs); diluting the reaction solution to 10 mL for later use.

### Embodiment 2

A nano coordination polymer comprises ditiocarb sodium, copper, and a hyaluronic acid, wherein the ditiocarb sodium and the copper form a polymer through coordination, and the polymer is wrapped by the hyaluronic acid through electrostatic interaction to form a core-shell structure.

A preparation method of the nano coordination polymer of the embodiment 2 comprises steps of:

(1) adding 6 mL 2 mM ditiocarb sodium (DTC) into a 20 mL beaker, adding 184 μL 20 wt % polyvinylpyrrolidone (PVP), and stirring at a room temperature for 2 min to obtain a mixed solution;

(2) using a 5 mL syringe to take 3 mL 2 mM CuCl₂ and dripping into the mixed solution of the step (1) with a constant flow pump at a dripping rate of 20 μL/min, thereby obtaining a ditiocarb sodium/copper nano coordination polymer (CuET NPs); diluting the reaction solution to 10 mL for later use:

(3) precisely weighing 12 mg hyaluronic acid (HA) and dissolving in 20 mL distilled water to prepare a 0.6 mg/mL HA solution; and

(4) adding 4 mL CuET NPs in a 20 mL beaker; stirring and using a 5 mL syringe to take 4 mL HA solution, and dripping into the beaker with the constant flow pump at a dripping rate of 100 μL/min; then stirring at the room temperature for 6 h to obtain a nano coordination polymer: CuET@HA NPs.

Experiment:

I. Particle size: the particle sizes of CuET NPs and CuET@HA NPs are measured, and the measurement method is: placing sample solutions in a Marlven Nano ZS instrument, and detecting the particle sizes with a dynamic light laser scattering method, wherein the measurement temperature is set to 25° C., and each sample is parallelly operated for 3 copies. FIG. 1 illustrates particle sizes and Zeta-potential changes of CuET NPs and CuET@HA NPs, which shows that the particle size of the nano coordination polymer CuET NPs is 98 nm and the Zeta-potential is +1.73 mV. The particle size of the coordination polymer CuET@HA NPs wrapped by the hyaluronic acid is increased to 125 nm, and the Zeta-potential is decreased to −23.2 mV.

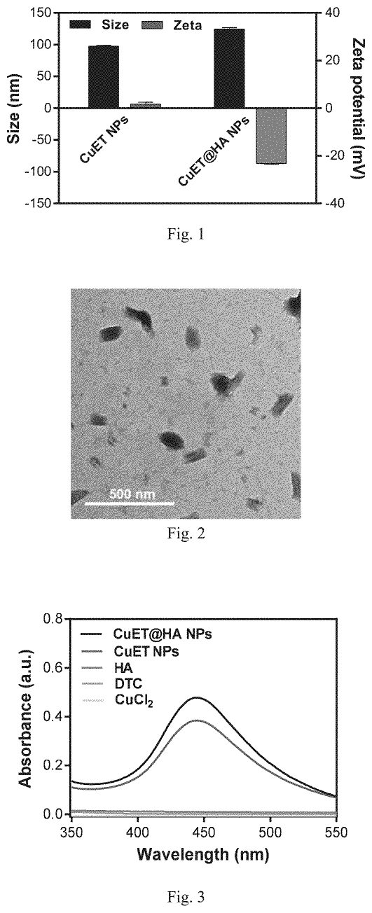

II. Morphology: the morphology of CuET@HA NP is observed, and a observing method is: dripping a sample on a 400 mesh copper net covered with carbon film, placing in a desiccator, and placing on a transmission electron microscope Titan G2-F20 for observing after being naturally dried. FIG. 2 is a transmission electron microscope image of CuET@HA NPs, which shows that the CuET@HA NPs of the present invention is an amorphous polymer under the transmission electron microscope.

III. Ultraviolet spectroscopy: UV spectrum scanning is performed on CuET NPs and CuET@&HA NPs, and a detecting method is: using distilled water as a blank control solution to detecting UV absorption spectra of CuET NPs and CuET@HA NPs. FIG. 3 is a UV absorption spectrum of CuET NPs and CuET@HA NPs, which shows that: DTC, HA and CuCl₂ have no obvious absorption peak at 350-550 nm; CuET NPs and CuET@HA NPs have plasmon resonance absorption peaks at about 444 nm.

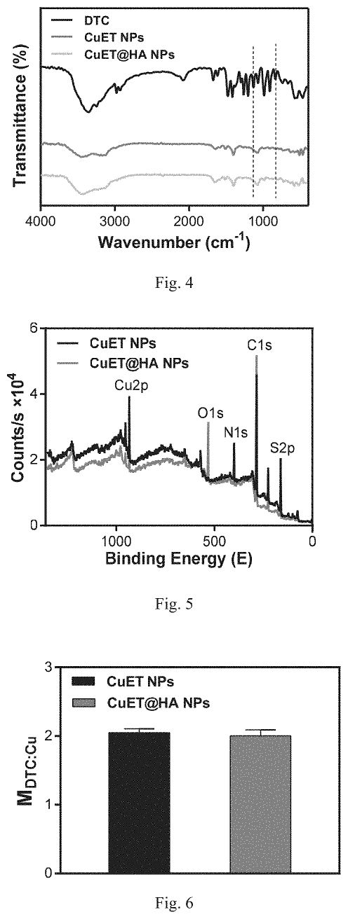

IV. Fourier near infrared spectroscopy: infrared spectra of DTC, CuET NPs and CuET@HA NPs are scanned. FIG. 4 is an infrared absorption spectrum of CuET NPs and CuET@HA NPs, which shows that DTC has C═S bond and C—S bond absorption peaks at 1128 cm⁻¹ and 834 cm⁻¹, respectively. At the same time C═S bond and C—S bond absorption peaks of CuET NPs and CuET@HA NPs disappear, proving that DTC is coordinated with Cu²⁺ through C═S bond and C—S bond.

V. X-ray photoelectron spectroscopy: X-ray photoelectron spectroscopies of CuET NPs and CuET@HA NPs are scanned. FIG. 5 is an X-ray photoelectron spectrogram of CuET NPs and CuET@HA NPs, which shows that there is no absorption peak of O element in the energy spectrum of CuET NPs, while the absorption peak of O element appears in the energy spectrum of CuET@HA NPs, which proves that CuET NPs has been successfully coated with HA.

VI: Molar ratio: FIG. 6 illustrates a molar ratio of DTC to Cu²⁺ in CuET NPs and CuET@HA NPs, which shows that the molar mass ratio of DTC and Cu²⁺ in CuET NPs and CuET@HA NPs is about 2:1, and encapsulation efficiency is 100%.

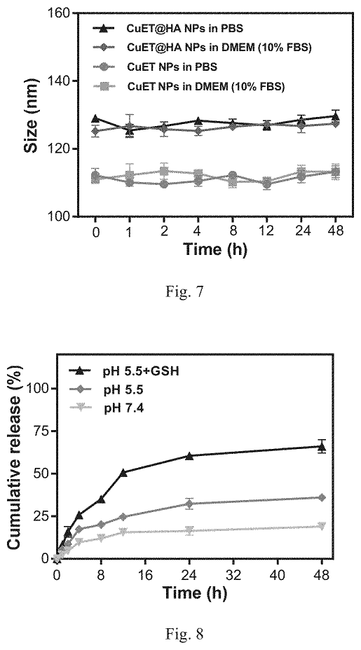

VII. Stability test: CuET NPs and CuET@HA NPs are placed in PBS and DMEM complete media containing 10% fetal bovine serum (FBS) at 37° C., respectively; and particle sizes are measured at different time points. FIG. 7 illustrates particle size changes of CuET NPs and CuET@HA NPs in PBS and DMEM complete media, which shows that the particle sizes of CuET NPs and CuET@HA NPs have no obvious changes, indicating good stability of CuET NPs and CuET@HA NPs in PBS solution and plasma.

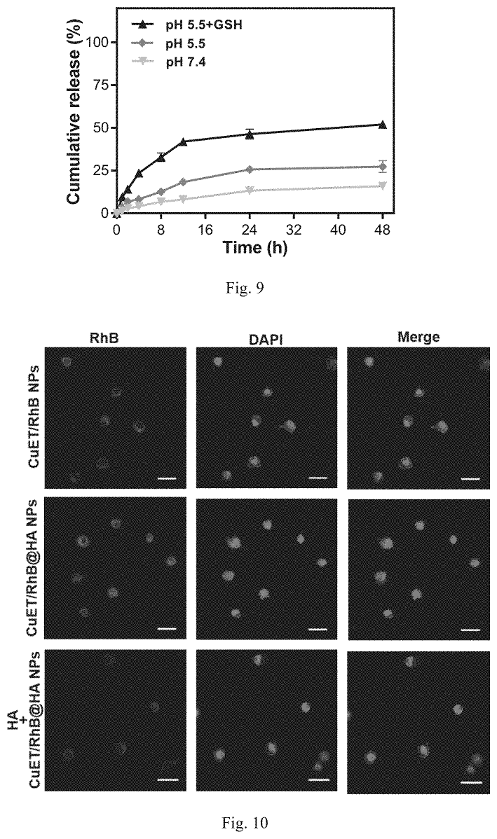

VIII. Cumulative release rate: diluting CuET NP by 4 times, and diluting CuET@HA NPs by 2 times, then adding 1 mL of each into a dialysis bag with a molecular weight cut-off of 3500 (CuET NP and CuET@HA NPs, 21 copies each), placing the dialysis bags containing nanoparticles in 50 mL centrifuge tubes containing release media, wherein the release media are: pH 7.4, pH 5.5, pH 5.5+10 mM GSH; taking out the solution in the dialysis bag at 1, 2, 4, 8, 12, 24, and 48 h, and measuring absorbance A at 444 nm; taking absorbance of unreleased nanoparticles as A₀, wherein cumulative release rate=(1−A/A₀)×10. FIGS. 8 and 9 are release curve diagrams of CuET NPs and CuET@HA NPs under different conditions, which show that both CuET NPs and CuET@HA NPs have certain acid sensitivity and strong glutathione responsiveness. Compared with CuET NPs, CuET@HA NPs has lower release rate, indicating that HA has a certain stabilizing and protecting effects on CuET NPs.

### Embodiment 3

Studying tumor targeting effect of the nano coordination polymers of embodiments 1 and 2:

(1) preparing Rhodamine B (RhB)-loaded nano coordination polymers CuET/RhB NPs and CuET/RhB@HA NPs, wherein specific steps are:

1.1. adding 6 mL 2 mM DTC into a 20 mL beaker, and adding 184 μL 20 wt % PVP and 188 μL 1 mM Rhodamine B (RhB); stirring at room temperature for 2 min; using a 5 mL syringe to take 3 mL 2 mM CuCl₂ and dripping with a constant flow pump at a dripping rate of 20 μL/min, and then stirring for 5 min to obtain CuET/RhB NPs; and

1.2. adding 4 mL CuET/RhB NPs in a 20 mL beaker, adding 4 mL HA solution and stirring at room temperature for 6 h to obtain CuET/RhB@HA NPs;

(2) taking logarithmically-grown MDA-MB-231 cells (human-derived triple-negative breast cancer cells, M231 cells, purchased from Xiangya Medical Experimental Center, Central South University), digesting and counting, and diluting to 2×10⁵ cells/mL cell suspension with an appropriate amount of DMEM complete medium; seeding in a 24-well plate with 2 mL per well, wherein a total of 3 wells are seeded; after 24 h of adherent culture, aspirating and discarding the medium, and washing with PBS for 3 times;

(3) adding 2 mL 5 mg/mL free HA (dissolved in DMEM without FBS) in one well, and adding 2 mL DMEM without FBS to the remaining 2 wells; after 4 h of incubation, aspirating and discarding the medium, and washing with PBS for 3 times:

(4) diluting CuET/RhB NPs and CuET/RhB@HA NPs to 100 nM (in terms of RhB) sample solutions with the DMEM medium (without FBS);

(5) adding 2 mL CuET/RhB NPs to a well without HA intervention, and adding CuET/RhB@HA NPs to the remaining two wells; after incubating at 37° C. for 4 h, aspirating and discarding the medium, and washing with PBS for 3 times:

(6) adding 1 mL paraformaldehyde to each well and fixing for 20 min in dark, aspirating and discarding supernatant, and washing with PBS for 3 times; and

(7) adding 0.5 mL 1 μg/mL DAPI to each well, and staining nucleus for 15 min in dark; aspirating and discard supernatant, and washing with PBS for 3 times; then observing fluorescence intensity of each well under a confocal laser microscope.

FIG. 10 shows cellular uptake of CuET@HA NPs, wherein CuET NPs and CuET@HA NPs with the fluorescent dye RhB are incubated with M231 cells for 4 h before fluorescence imaging. M231 cells are pretreated with 15 mg/mL HA for 4 h, and then NPs are incubated with the cells for 4 h before observing by the laser confocal microscope. A DAPI channel indicates that the nucleus is stained with blue fluorescence, an RhB channel indicates that NPs are labeled with red fluorescence, and Merged superimposes the DAPI and RhB channels. HA+ means free HA pretreatment, and scale=50 μm.

It can be seen from FIG. 10 that after the two nano preparations are incubated with M231 cells for 4 h, obvious red fluorescence exists in the cells under the fluorescence microscope, indicating that nanoparticles are taken up by the cells. The well fluorescence of CuET/RhB@HA NPs is stronger than that of CuET/RhB NPs. However, after 4 h of pretreatment with free HA, red color is significantly reduced, which indicates that the hyaluronic acid functionalized nano coordination polymer has an active targeting effect on tumor cells, and being wrapped by the hyaluronic acid can enhance uptake of the nano coordination polymer by the tumor cells.

### Embodiment 4

Studying cytotoxicity of the nano coordination polymers of the embodiments 1 and 2 to the tumor cells:

(1) performing trypsin digestion to the logarithmically-grown M231 cells and HEK-293 cells, and diluting into 5×10⁴ cells/mL with DMEM medium containing 10% fetal bovine serum; seeding in a 96-well plate with 100 μL per well; after incubating for 24 h in a carbon dioxide incubator (37° C., 5% CO₂, saturated humidity), discarding the culture medium;

(2) adding 100 μL per well DTC, CuET NP and CuET@HA NPs diluted to different concentrations with the culture medium (concentrations are 16, 32, 64, 125, 250, 500, 1000 and 2000 nM in terms of DTC), repeating 6 wells for each concentration and incubating for 48 h;

(3) add 10 μL MTT solution (5 mg/mL) to each well, then incubating for 4 h before terminating culture; and aspirating and discarding supernatant; and

(4) adding 150 μL DMSO solution to each well, placing on a shaker and shaking at low speed for 10 min to completely dissolve crystals, and measuring absorbance (OD) at 570 nm with a microplate reader.

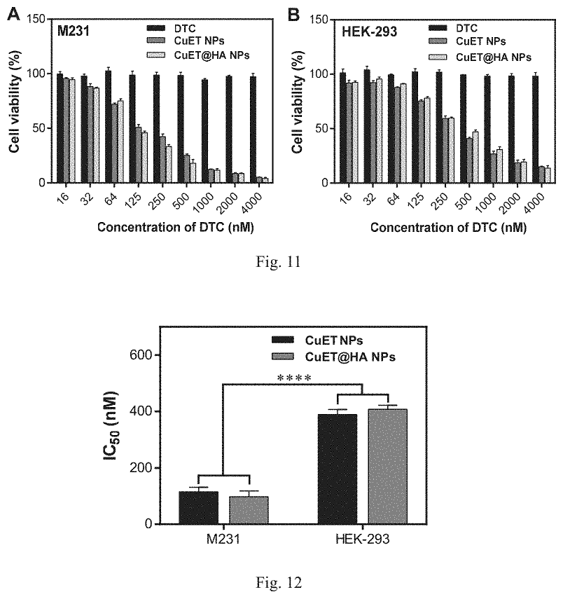

FIG. 11 illustrates cell viability of CuET NPs and CuET@HA NPs measured by an MTT method after incubating with cells for 48 h. Part A shows M231 cells and Part B shows HEK-293 cells. It can be seen that the cell viability of CuET NPs and CuET@HA NPs of the present invention are dose-dependent.

FIG. 12 is IC₅₀ results. Data are expressed as mean±standard deviation (n=6). It can be seen from IC₅₀ values that the cytotoxicity of CuET NP and CuET@HA NPs to M231 cells is 3 times of that to HEK-293.

It can be seen from results of the cytotoxicity experiment that the nano coordination polymers of the embodiments 1 and 2 of the present invention can inhibit tumor cell proliferation and promote cell apoptosis, but are less toxic to normal macrophages, which means the coordination polymers of the present invention have certain anti-tumor efficacy in-vitro and can be used as drugs to inhibit tumor growth.

### Embodiment 5

Studying cellular effects of the nano coordination polymers on the tumor cells:

(1) seeding M231 cells in a 6-well plate at 4×10⁵/well, and incubating with CuET NPs at concentrations of 0.1, 0.2, 0.5, and 1 μM for 24 h;

(2) lysing M231 cells with Western lysis buffer, collecting protein samples in the cells, and determining protein concentration of the protein samples;

(3) preparing SDS-PAGE gel, adding an appropriate amount of concentrated SDS-PAGE protein loading buffer to the collected protein samples, and heating at 100° C. or boiling water bath for 3-5 min to fully denature the protein;

(4) after cooling to room temperature, directly loading the protein sample into sample wells of the SDS-PAGE gel for electrophoresis, and stopping electrophoresis when bromophenol blue reaches bottom of the gel:

(5) using a PVDF membrane for transfer with a Bio-Rad standard wet transfer device, and then adding 5% skim milk and sealing at room temperature for 1 h;

(6) aspirating a sealing solution, adding diluted primary antibody, and incubating overnight at room temperature; recovering the primary antibody, adding Western washing solution, and washing for 3 times;

(7) diluting horseradish peroxidase (HRP)-labeled secondary antibody with Western secondary antibody diluent in an appropriate ratio; aspirating the washing solution, adding the diluted secondary antibody, and incubating at the room temperature for 1 h; washing for 3 times; and

(8) finally detecting protein with an ECL reagent such as BeyoECL Plus (P0018).

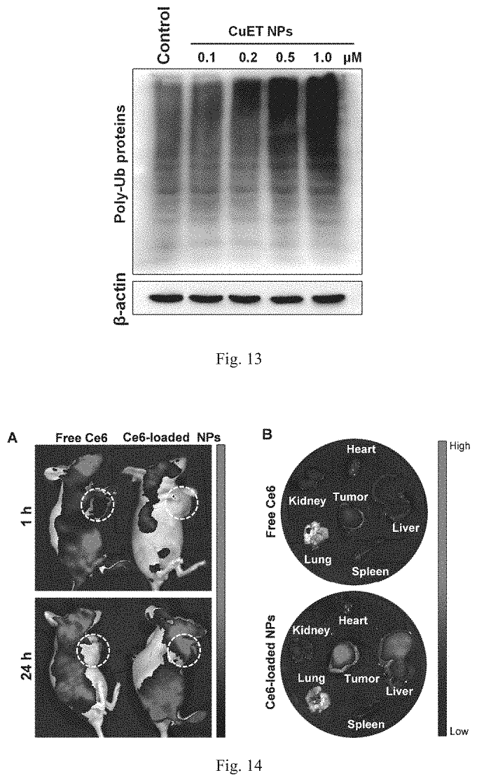

FIG. 13 illustrates results of ubiquitinated protein expression after incubating CuET NPs having concentrations of 0.1, 0.2, 0.5 and 1 μM for 24 h with M231 cells. *P<0.05, **P<0.01. ***P<0.001. It can be seen that as the concentration of CuET NPs increases, ubiquitinated proteins gradually accumulate, indicating that the nano coordination polymer of the present invention can cause protein ubiquitination in the tumor cells.

### Embodiment 6

Studying in-vivo distribution of the nano coordination polymers:

(1) establishing a tumor-bearing nude mouse model: collecting logarithmically-grown M231 cells and dispersing in PBS at a cell density of 1×10⁷/100 μL; mixing with Matrigel with an equal volume, and injecting into BALB/c nude mice (female, 6 weeks old) under armpit, wherein the female BALB/c nude mice, 6 weeks old, are purchased from Changzhou Cavins Laboratory Animal Co., Ltd.;

(2) treating: when the mouse tumor grows to 200 mm³, injecting free Ce6 and Ce6-loaded CuET@HA NPs (Ce6, 2.5 mg/kg) into tail vein of the mice; and

(3) detecting: anesthetizing the mice at 1 h and 24 h after injection, and imaging the mice by an in-vivo imaging system; after 24 h of in-vivo imaging, sacrificing the mice; taking out heart, liver, spleen, lung, kidney and tumor, and imaging with the imaging system.

FIG. 14 illustrates in-vivo distribution of the drug, wherein the nude mice are injected with free Ce6 and Ce6-loaded CuET@HA NPs at the tail vein, and are imaged at different time points. Part A is fluorescence distribution in mice at 1 h and 24 h after tail intravenous injection; part B is fluorescence distribution of heart, liver, spleen, lung, kidney and tumor after the mice were sacrificed 24 h after the injection.

It can be seen from part A that: at 1 h, fluorescence intensity of the mouse injected with free fluorescein is stronger than that of the mouse injected with nanoparticles, and the fluorescence intensities of the two mice are opposite after 24 h. After 24 h, the fluorescence intensity of a tumor site of the mouse injected with the nanoparticles is stronger than that of other sites, while the mouse injected with the free fluorescein does not have such trend. Referring to part B, among isolated tumors, the fluorescence intensity of the tumors of the mouse injected with the nanoparticles is significantly stronger than that of the mouse injected with the free fluorescein, indicating that the nano coordination polymer of the present invention can accumulate in tumor sites and has tumor targeting property.

### Embodiment 7

In-vivo anti-tumor activity of the nano coordination polymers:

The mice are treated according to the method of the embodiment 5. When the tumors of the tumor-bearing mice grow to about 200 mm³, the mice are randomly divided into 4 groups (n=6), and each group is injected on day 0, 3, 6, and 9 with PBS, free DTC, CuET NPs, CuET@HA NPs (DTC: 1 mg/kg); the mice are weighed every two days and tumor volumes are measured with a vernier caliper until day 14; then comparing anti-tumor efficiency of each group through relative volumes of the tumors of each group. Tumor volume calculation formula: V=length×width²/2.

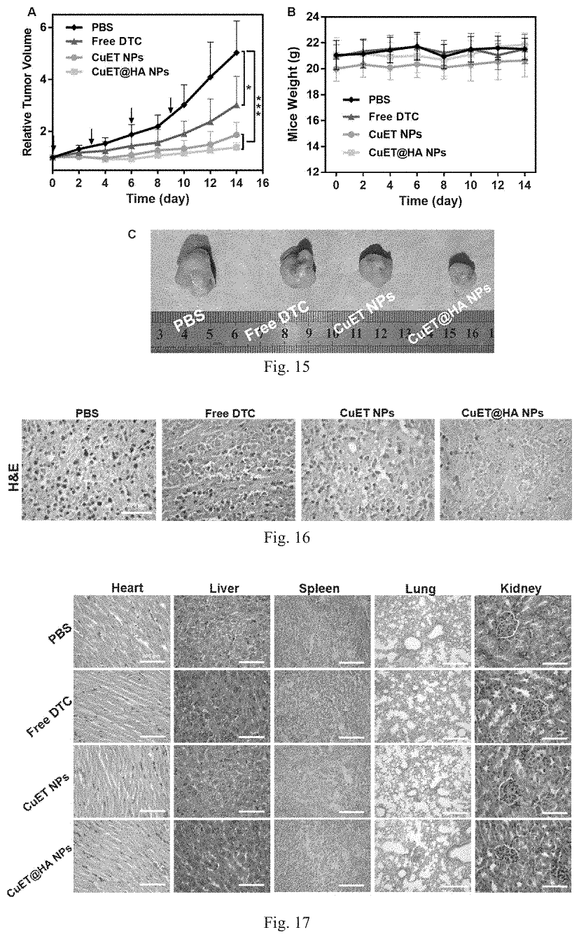

FIG. 15 shows tumor volume change curves after tail intravenous with PBS, free DTC, CuET NPs and CuET@HA NPs on day 0, 3, 6 and 9. FIG. 15A is a graph of tumor volume change in each group; part A is a graph of tumor volume change in each group; part B is a graph of weight changes of mice in each group; part C is an image of solid tumors in each group on day 14, wherein 1 is PBS: 2 is free DTC: 3 is CuET NPs; and 4 is CuET@HA NPs.

FIG. 16 is H&E staining images of tumor tissues in each group. Data are expressed as mean standard deviation (n=6), *P<0.05, **P<0.01, ***P<0.001.

It can be seen from part A and part C of FIG. 15 that, compared with the PBS group and free DTC, both CuET NPs and CuET@HA NPs (DTC: 1 mg/g) have certain anti-tumor effects, wherein tumor inhibition effect of CuET@HA NPs is slightly stronger than that of CuET NPs group. It can be seen from part B of FIG. 15 that the body weight of the mice in each group is not changed significantly during administration period, indicating that the nano coordination polymer can significantly inhibit tumor growth and has a strong anti-tumor effect.

### Embodiment 8

In-Vivo Safety of the Nano Coordination Polymers:

The mice are treated according to the method of the embodiment 5. Four groups of mice are sacrificed on day 14 after administration. Heart, liver, spleen, lung and kidney are taken out, washed with physiological saline, dried by filter paper, and fixed with 4% paraformaldehyde for 24 h. The tissues are embedded in paraffin, sectioned, and HE stained to observe pathological changes with an optical microscope.

FIG. 17 illustrates pathological sections of heart, liver, spleen, lung and kidney of each group of mice. Scale=100 μm. Compared with PBS group, the other three groups had no obvious pathological changes, indicating that the nano coordination polymer has good in-vivo safety.

Obviously, the above embodiments are merely examples for clear description, and are not intended to be limiting. For those of ordinary skill in the art, other modifications or changes in different forms can be made based on the above description. It is unnecessary and impossible to list all implementations. The obvious modifications or changes based on the above description are all within the protection scope of the present invention.

What is claimed is:

1. A nano coordination polymer, comprising: ditiocarb sodium and copper, wherein the ditiocarb sodium and the copper form a polymer through coordination.

2. The nano coordination polymer, as recited in claim 1, wherein a molar concentration ratio of the ditiocarb sodium and the copper is 2:1.

3. The nano coordination polymer, as recited in claim 1, wherein nano coordination polymer is wrapped by a targeting ligand through electrostatic interaction to form a core-shell structure.

4. The nano coordination polymer, as recited in claim 2, wherein nano coordination polymer is wrapped by a targeting ligand through electrostatic interaction to form a core-shell structure.

5. The nano coordination polymer, as recited in claim 3, wherein the targeting ligand is a hyaluronic acid, synthetic polypeptide, a folate-modified hydrophilic polymer, or a tumor-targeted nucleic acid aptamer.

6. The nano coordination polymer, as recited in claim 4, wherein the targeting ligand is a hyaluronic acid, synthetic polypeptide, a folate-modified hydrophilic polymer, or a tumor-targeted nucleic acid aptamer.

7. A preparation method of a nano coordination polymer, comprising steps of:
S1: mixing ditiocarb sodium with a stabilizer to obtain a mixed solution, and dripping CuCl₂ into the mixed solution to obtain a polymer; and
S2: dripping a targeting ligand, which is in a solution form, into the polymer; then stirring to obtain the nano coordination polymer.

8. The preparation method, as recited in claim 7, wherein the stabilizer is polyethylene glycol, polyvinyl alcohol, poly vinylpyrrolidone or Tween.

9. The preparation method, as recited in claim 7, wherein a concentration of the stabilizer is 0.4 wt %-1 wt %.

10. A method for preparing a tumor targeted therapy drug, comprising adding a nano coordination polymer, as recited in 1, into the tumor targeted therapy drug.
